(19) United States
(12) Patent Application Publication
(10) Pub. No.: US 2012/0029405 A1
(43) Pub. Date: Feb. 2, 2012

(54) Scented And Antiseptic Adhesive Bandage

(76) Inventors: Anthony Cataldi, Staten Island, NY (US); Linda Cataldi, Staten Island, NY (US)

(21) Appl. No.: 13/035,028

(22) Filed: Feb. 25, 2011

Related U.S. Application Data
(60) Provisional application No. 61/369,680, filed on Jul. 31, 2010.

(51) Int. Cl. A61F 13/02

(52) U.S. Cl. 602/48

(57) ABSTRACT

A healing bandage for covering a skin wound comprising: an adhesive backing, where said backing includes a sticky layer on an underside to adhere to the skin of a wearer and a smooth top side with a scented coating; a gauze pad centrally attached to the underside of the adhesive backing; and an antiseptic layer on the surface of the gauze pad. The antiseptic layer on the gauze pad covers the skin wound and the scented coating releases a scent. The backing may be comprised of plastic or cloth and include a plurality of holes. The scent may include at least one of a fruity, floral, tropical or fresh.

### CROSS REFERENCE TO OTHER APPLICATIONS

This application claims priority to U.S. Provisional Application Ser. No. 61/369,680 filed on Jul. 31, 2010.

### BACKGROUND OF THE INVENTION

1. Field of the Invention

The present invention relates to a scented adhesive bandage including an antiseptic material on a centralized gauze strip.

2. Description of Related Art

Adhesive bandages are a common item found in most medicine cabinets and first aid kits. Bandages allow a person to comfortably wear a sterile piece of gauze without having to continuously replace new gauze and tape. Adhesive bandages come in a wide variety of textures, colors and shapes. Some bandages are made from plastic for waterproof characteristics while others are made from a flexible cloth for greater flexibility and movement. Some bandages are specifically designed to be attractive to kids, like wearing a sticker, which come in a variety of textures, shapes and colors. Children enjoy wearing these decorative bandages because the designs may be of their favorite cartoon character, animal or even sport.

Many times an antiseptic or ointment is placed directly on a wound and then covered with an adhesive bandage. Commonly used antiseptics are products like Neosporin®, Bactine® or others which may sell as creams or gels to place directly on the wound. The antiseptic allows the wound to heal faster and reduces scarring on the surface of the skin. Often times, after the bandage is placed, the antiseptic squishes out from under the bandage, requiring continuous replacement. Alternatively, the antiseptic may cause the adhesive portion of the bandage to lose its sticky qualities causing the bandage to come loose or even fall off

It would be desirable in the art to have a bandage which combines a way to apply antiseptic or ointment with an adhesive bandage. It would also be beneficial for the bandages to have scents to be further appealing to children.

### SUMMARY OF THE INVENTION

The present invention relates to a healing bandage for covering a skin wound comprising: an adhesive backing, where said backing includes a sticky layer on an underside to adhere to the skin of a wearer and a smooth top side with a scented coating; a gauze pad centrally attached to the underside of the adhesive backing; and an antiseptic layer on the surface of the gauze pad. The antiseptic layer on the gauze pad covers the skin wound and the scented coating releases a scent. The backing may be comprised of plastic or cloth and include a plurality of holes. The scent may include at least one of a fruity, floral, tropical or fresh.

### BRIEF DESCRIPTION OF THE DRAWINGS

The advantages and features of the present invention will become better understood with reference to the following detailed description and claims taken in conjunction with the accompanying drawings, wherein like elements are identified with like symbols, and in which:

FIG. 1 depicts a perspective view of an underside of a healing bandage in accordance with an exemplary embodiment of the present invention;

FIGS. 2A and 2B depict perspective views of an underside of a healing bandage in accordance with alternative embodiments of the present invention; and

FIGS. 3A, 3B, 3C and 3D depict perspective views of a topside of a healing bandage in accordance with alternative embodiments of the present invention.

Like reference numerals refer to like parts throughout the description of several views of the drawings.

### DETAILED DESCRIPTION OF THE DRAWINGS

The present invention relates to adhesive bandages with a layer of antiseptic to aid in healing and scar reduction, while providing a scented top surface for appeal to children and adults alike. The bandages may be available in a variety of shapes, sizes, colors, textures and scents for a decorative yet functional feature. The antiseptic used may be an aloe gel or aloe extracted cream selected for its healing qualities and comfortable feel. The bandages may be used on various types of wounds ranging from cuts and blisters to burns and scrapes, all the while providing a healing touch with decorative appeal.

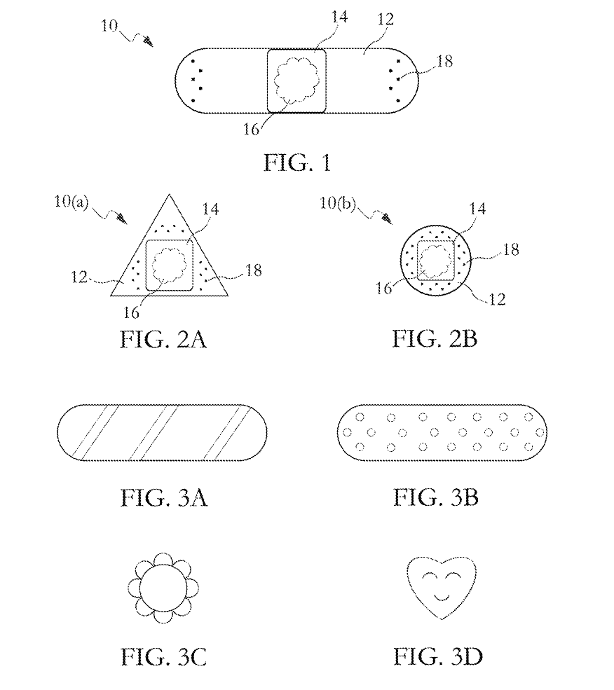

Turning now descriptively to the drawings, referring to FIG. 1, a perspective view of an underside of a healing bandage (10) is shown in accordance with an exemplary embodiment of the present invention. The healing bandage (10) may include an adhesive backing (12) and a gauze pad (14). The gauze pad (14) may be centrally located against the adhesive backing (12), with portions of the adhesive backing (12) extending around the gauze pad (14) to secure the healing bandage (10) to a person's skin. On the underside of the adhesive backing (12) may be a thin layer of glue or sticky material to enable the healing bandage (10) to stick to the skin. The adhesive backing (14) may be comprised of a plastic material, a cloth strip or any other type of material commonly used for a bandage. On a top surface of the adhesive backing (12), there is no adhesive material so that the healing bandage (10) is smooth to the touch. Rather there may a thin scented coating or layer on the top side of the adhesive backing (12) to give the healing bandage (10) a pleasing odor. The odor may be fruity, floral, tropical, fresh or a variety of other scents depending on the preferences of the maker and user. The adhesive backing (12) may have a plurality of holes (18) (hereinafter holes) to enable the skin to breath and wick moisture away when the healing bandage (10) is in place.

Accordingly, the gauze pad (14) may be a sterile cotton lining to prevent further infection or damage to the wound, and may absorb excess liquids like pus or blood coming from the wound. The gauze pad may contain an antiseptic layer (16) to hold against a wound on the patch of skin. The antiseptic layer (16) may be made from an aloe vera gel, liquid, aloe extracted cream or another type of antibacterial or disinfecting substance to aid in the healing of the wound. When the healing bandage (10) is placed over the wound, the adhesive backing sticks to the surrounding skin so that the gauze pad (14) with the antiseptic layer (16) securely rest above the wound. By securing the antiseptic layer (16) against the wound, the time needed for the wound to heal is reduced and scarring is less likely to occur. The material comprising the adhesive backing (12) may be flexible and waterproof to enable a person to wear the healing bandage (10) while bathing and during activities without risk of undesired removal.

Referring to FIGS. 2A and 2B, perspective views of an underside the healing bandage (10) are shown in accordance with an alternative embodiments of the present invention. FIG. 2A shows a healing bandage in the shape of a triangle. The triangular shape of the healing bandage 10(a) includes the adhesive backing (12), the gauze pad (14), the antiseptic layer (16) and the holes (18). FIG. 2B illustrates a circular healing bandage 10(b) which may be used to cover smaller wounds or in areas with lesser skin surface for attachment. The triangular and circular shaped healing bandages 10(a) and 10(b) may function the same as the healing bandage (10) shown in FIG. 1, but with a shape which may be more aesthetically pleasing to a wearer.

Referring to FIGS. 3A, 3B, 3C and 3D, perspective views of a topside of various healing bandages are shown in accordance with alternative embodiments of the present invention. FIG. 3A and FIG. 3B illustrate two different designs on an exposed surface of the adhesive backing The designs may be stripes, polka dots, various colors, cartoon characters, sport activities, food, animals, faces or a plurality of different patterns or pictures desired to be worn by the wearer. FIGS. 3C and 3D illustrate a couple of shapes which may be aesthetically pleasing to children when wearing the healing bandage. The shapes may also be, but not limited to, animals, food, geometric shapes, people, toys, letters, numbers, etc. The various designs and shapes of the healing bandages may offer a fun alternative to non-decorated bandages for children or adults who want decorative pieces on their skin.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A healing bandage for covering a skin wound comprising;
an adhesive backing, where said backing includes a sticky layer on an underside to adhere to the skin of a wearer and a smooth top side with a scented coating;
a gauze pad centrally attached to the underside of the adhesive backing; and
an antiseptic layer on the surface of the gauze pad.

2. The healing bandage according to claim 1, wherein the antiseptic layer on the gauze pad covers the skin wound and the scented coating releases a scent.

3. The healing bandage according to claim 1, where the backing is comprised of plastic or cloth.

4. The healing bandage according to claim 1, where the adhesive backing includes a plurality of holes.

5. The healing bandage according to claim 2, where the scent includes at least one of fruity, floral, tropical or fresh.

6. The healing bandage according to claim 1, where the gauze pad includes a sterile cotton lining.

7. The healing bandage according to claim 1, where the antiseptic layer is mad from an aloe vera substance in the form of a gel, liquid or cream.
